(19) United States
(12) Patent Application Publication
(10) Pub. No.: US 2006/0193925 A1
(43) Pub. Date: Aug. 31, 2006

(54) Side effect-relieving agents and/or hypoglycemic effect enhancers for thiazolidine compounds

(75) Inventors: Yasuo Morimoto, Osaka (JP); Tomoko Maegawa, Osaka (JP)

Correspondence Address: FOLEY AND LARDNER LLP;SUITE 500, 3000 K STREET NW, WASHINGTON, DC, 20007, US

(73) Assignee: KANEBO, LIMITED

(21) Appl. No.: 11/410,884

(22) Filed: Apr. 26, 2006

Related U.S. Application Data
(62) Division of application No. 10/772,587, filed on Feb. 6, 2004.
Application No. 10/772,587 is a continuation-in-part of application No. PCT/JP02/07764, filed on Jul. 30, 2002.

(30) Foreign Application Priority Data
Aug. 7, 2001 (JP) 2001-238898
Feb. 19, 2002 (JP) 2002-041826

(51) Int. Cl. A61K 33/06; A61K 36/185; A61K 36/48

(52) U.S. Cl. 424/696; 424/725; 424/757

(57) ABSTRACT

The present invention relates to a side effect-relieving agent for thiazolidine compounds which comprises a crude drug mixture as an active ingredient comprising Ephedrae Herba, Glycyrrhizae Radix and Gypsum Fibrosum each of which is in a form of ground powders, extracts or mixtures of powders and extracts; and a hypoglycemic effect enhancer for thiazolidine compounds which comprises a crude drug mixture as an active ingredient comprising Ephedrae Herba, Glycyrrhizae Radix and Gypsum Fibrosum each of which is in a form of ground powders, extracts or mixtures of powders and extracts. Using the preparation according to the present invention, body weight gain, which is a side effect of thiazolidine compounds, can be relieved and the hypoglycemic effect of the thiazolidine compounds can be enhanced.

The present application is a divisional of U.S. application Ser. No. 10/772,587, filed Feb. 6, 2004, which is a continuation-in-part of PCT/JP02/07764, filed Jul. 30, 2002, the entire contents of which are incorporated herein by reference.

### TECHNICAL FIELD

The present invention relates to a side effect-relieving agent and/or a hypoglycemic effect enhancer for thiazolidine compounds. More particularly, the present invention relates to a side effect-relieving agent for thiazolidine compounds which comprises a crude drug mixture as an active ingredient comprising Ephedrae Herba, Glycyrrhizae Radix and Gypsum Fibrosum each of which is in a form of ground powders, extracts or mixtures of powders and extracts; and a hypoglycemic effect enhancer for thiazolidine compounds which comprises a crude drug mixture as an active ingredient comprising Ephedrae Herba, Glycyrrhizae Radix and Gypsum Fibrosum each of which is in a form of ground powders, extracts or mixtures of powders and extracts.

### BACKGROUND ART

Currently, there are about 7 million diabetics in Japan, and the total of the current and potential diabetics is estimated to number approximately 14 million. Most of them are type 2 diabetics who manifest and develop their symptoms on the basis of insulin resistance attributable to their lifestyles such as overeating and insufficient physical exercise as well as to their genetic predisposition.

Insulin resistance which is a feature of type 2 diabetics is frequently accompanied with obesity, especially that caused by the accumulation of-visceral fat, and in many cases it is accompanied with hyperlipidemia, hypertension and the like at the same time.

To cope with this situation, various drugs for oral administration have been developed. For example, sulfonylurea drugs which stimulate the secretion of insulin by acting on pancreatic β-cells, biguanide drugs which suppress glycogenesis in the liver, drugs which suppress the absorption of glucose from the intestine by inhibiting the intestinal tract digestive enzyme disaccharidases, or thiazolidine compounds which lower the blood glucose levels by directly improving insulin resistance have been known. These drugs have been widely used in clinical settings.

Thiazolidine compounds, ligands of the intranuclear receptor PPAR (a peroxisome proliferator activated receptor) γ, have attracted attention as novel therapeutic agents for diabetes which have been developed recently, and the thiazolidine compounds lower the blood glucose levels by improving insulin resistance and some effects have been clinically observed (Nippon Rinsho, vol. 57, No. 3, pp. 688-694, 1999).

Although the thiazolidine compounds are remarkably effective, body weight gain and body fat gain have often been observed by long-term administration in effective cases, accompanied by a problem that hypoglycemic effect of the thiazolidine compounds was reduced (J. Japan Diab. Soc., vol. 44, No. 4, pp. 323-327, 2001).

To approach this problem, drugs which suppress body weight gain induced by thiazolidine compounds are also known. For example, WO93/3724 discloses that 3-guanidinopropionic acid (3-GPA) suppresses body weight gain induced by pioglitazone in a dose-dependent manner in KKA^{y} mice, obese diabetic animals. In addition, voglibose, an inhibitor of disaccharidase, is known to control body weight gain induced by pioglitazone in Wistar fatty rats, obese diabetic animals (Yakuri to Chiryo, vol. 25, No. 2, pp. 355-361, 1997).

However, the duration of administration of the above-mentioned drug and pioglitazone in combination was only 2 weeks in both studies, and it has not been elucidated whether the above-mentioned drugs control body weight gain induced by pioglitazone and prevent the reduction of hypoglycemic effects.

At the same time, no side effect-relieving agents and/or hypoglycemic effect enhancers for thiazolidine compounds comprising crude drugs as an active ingredient have been known.

Therefore, the present inventors made various studies for the purpose of finding a drug which is capable of relieving side effects of thiazolidine compounds, i.e. body weight gain, and enhancing the hypoglycemic effect of thiazolidine compounds.

### DISCLOSURE OF THE INVENTION

As a result of various studies, the present inventors found that a crude drug mixture comprising Ephedrae Herba, Glycyrrhizae Radix and Gypsum Fibrosum each of which is in a form of ground powders, extracts or mixtures of powders and extracts is capable of relieving the side effects of thiazolidine compounds, i.e., body weight gain, and enhancing the hypoglycemic effect of thiazolidine compounds, and they accomplished the present invention.

That is, the present invention is a side effect-relieving agent for thiazolidine compounds which comprises a crude drug mixture as an active ingredient comprising Ephedrae Herba, Glycyrrhizae Radix and Gypsum Fibrosum each of which is in a form of ground powders, extracts or mixtures of powders and extracts; and a hypoglycemic effect enhancer for thiazolidine compounds which comprises a crude drug mixture as an active ingredient comprising Ephedrae Herba, Glycyrrhizae Radix and Gypsum Fibrosum each of which is in a form of ground powders, extracts or mixtures of powders and extracts.

### BEST MODE FOR CARRYING OUT THE INVENTION

The Ephedrae Herba to be used in the present invention is the terrestrial stem of Ephedra sinica Stapf or other plants of the same genus (Ephedraceae), and specific examples include the one listed in the Guide to Japanese pharmacopeia, 13th edition (published by Hirokawa Shoten, 1996, hereinafter referred to as JP Guide), pages D-1017 to D-1021.

The Glycyrrhizae Radix to be used in the present invention is the root and the stolon, sometimes those that the periderm is removed (peeled Glycyrrhizae Radix), of Glycyrrhiza uralensis Fisher, Glycyrrhiza glabra Linne or other plants of the same genus (Leguminosae) and specific examples include the one listed in JP Guide, pages from D-227 to D-236.

Gypsum Fibrosum to be used in the present invention is natural hydrous calcium sulfate, and specific examples include the one listed in JP Guide, pages from D-563 to D-565.

Thiazolidine compounds include pioglitazone, troglitazone, rosiglitazone and pharmaceutically acceptable salts thereof. Of these compounds and salts thereof, pioglitazone hydrochloride and rosiglitazone maleate are more preferable.

Pioglitazone and pharmaceutically acceptable salts thereof are obtained according to the method of the preparation disclosed in Japanese Patent Laid-Open Publication (KOKAI) No. 22636/1980.

Troglitazone and pharmaceutically acceptable salts thereof are obtained according to the method of the preparation disclosed in Japanese Patent Laid-Open Publication (KOKAI) No. 51189/1985.

Rosiglitazone and pharmaceutically acceptable salts thereof are obtained according to the method of the preparation disclosed in Japanese Patent Laid-Open Publication (KOKAI) No. 131169/1989.

Preferably, a thiazolidine compound is used as a pharmaceutical preparation in which the compound is formulated (hereinafter referred to as a thiazolidine compound formulated preparation).

A side effect-relieving agent and/or a hypoglycemic effect enhancer for thiazolidine compounds of the present invention (hereinafter referred to as a medicament of the present invention) can be used as a mixture of ground crude drug powders of the above-described Ephedrae Herba, Glycyrrhizae Radix and Gypsum Fibrosum. The medicament of the present invention can also be used as a mixture of extracts described below. Further, the medicament of the present invention can be used as a mixture of ground crude drug powders and extracts, or extracts of the mixture of ground crude drug powders of the above-described Ephedrae Herba, Glycyrrhizae Radix and Gypsum Fibrosum can be used.

The medicament of the present invention can be used as a crude drug preparation containing Ephedrae Herba, Glycyrrhizae Radix and Gypsum Fibrosum each of which is in a form of ground powders, extracts or mixtures of powders and extracts. Such a crude drug preparation includes Bofu-tsusho-san (Fang-Feng-Tong-Shen-San), Goko-to (Wo-Hu-Tang), Makyo-kanseki-to (Ma-Xing-Gan-Shi-Tang) or Eppi-ka-jutsu-to (Yue-Bi-Jia-Zhu-Tang).

More preferably, the medicament of the present invention is used as these crude drug preparations.

A Bofu-tsusho-san extract used in the present invention is generally used as a concentrated extract or a dry extract powder obtained from mixed crude drugs usually comprising in weight ratio of 1.2 each of Angelicae Radix, Paeoniae Radix, Cnidii Rhizoma, Gardeniae Fructus, Forsythiae Fructus, Menthae Herba, Schizonepetae Spica, Saposhnikoviae Radix and Ephedrae Herba, 2.0 each of Atractylodis Rhizoma, Platycodi Radix, Scutellariae Radix, Glycyrrhizae Radix and Gypsum Fibrosum, 3.0 of Talcum, 0.3 to 0.4 of Zingiberis Rhizoma, 1.5 of Rhei Rhizoma and 0.7 to 1.5 of mirabilite (Senmei-ron). Anhydrous mirabilite or dried sodium sulfate may be used instead of mirabilite.

The above Bofu-tsusho-san extract can be prepared as follows. First, to the above-mentioned mixed crude drugs, water, a water-soluble organic solvent or a mixture thereof in an amount of 5 to 25 folds, preferably 8 to 20 folds in weight ratio is added, and this mixture is usually heated for 30 minutes to 2 hours at 80 to 100° C. to obtain the Bofu-tsusho-san extract by decoction. With respect to the above-mentioned water-soluble organic solvents, ethanol is preferred.

Further, the decoction is filtered or centrifuged to remove the decoction residue and then to make a concentrated extract using a conventional concentration means, for example, concentration under reduced pressure, or a dry extract powder using a conventional drying method, for example, drying under reduced pressure, spray drying or freeze-drying.

A Goko-to extract to be used in the present invention is generally used as a concentrated extract or a dry extract powder obtained from mixed crude drugs usually comprising in weight ratio of 4.0 each of Ephedrae Herba and Armeniacae Semen, 2.0 of Glycyrrhizae Radix, 10.0 of Gypsum Fibrosum and 3.0 of Mori Cortex (Manbyo-Kaisyun).

The Goko-to extract can be prepared in the same manner as used for the above-described Bofu-tsusho-san extract.

A Makyo-kanseki-to extract to be used in the present invention is generally used as a concentrated extract or a dry extract powder obtained from mixed crude drugs usually comprising in weight ratio of 4.0 each of Ephedrae Herba and Armeniacae Semen, 2.0 of Glycyrrhizae Radix and 10.0 of Gypsum Fibrosum (Shokan-ron, Kinki-yoryaku).

The Makyo-kanseki-to extract can be prepared in the same manner as used for the above-described Bofu-tsusho-san extract.

An Eppi-ka-jutsu-to extract to be used in the present invention is generally used as a concentrated extract or a dry extract powder obtained from mixed crude drugs usually comprising in weight ratio of 6.0 of Ephedrae Herba, 2.0 of Glycyrrhizae Radix, 8.0 of Gypsum Fibrosum, 3.0 of Zizyphi FruCtU3, 4.0 of Atractylodis Rhizome or Atractylodis Lanceae Rhizoma, and 0.8 to 1.0 of Zingiberis Rhizoma (Kinki yoryaku).

The Eppi-ka-jutsu-to extract can be obtained in the same manner as used for the above-described Bofu-tsusho-san extract.

A medicament of the present invention comprises, on the basis of 1 part by weight of a thiazolidine compound, 0.1 to 5000 parts by weight, preferably 0.5 to 4000 parts by weight, more preferably 1 to 3000 parts by weight, of a crude drug mixture comprising Ephedrae Herba, Glycyrrhizae Radix and Gypsum Fibrosum each of which is in a form of ground powders, extracts or mixtures of powders and extracts.

Usually, the formulation ratio for Ephedrae Herba, Glycyrrhizae Radix and Gypsum Fibrosum in the medicament of the present invention is, on the basis of 1 part by weight of Ephedrae Herba, 0.1 to 3 parts by weight of Glycyrrhizae Radix and 0.5 to 5 parts by weight of Gypsum Fibrosum, preferably 0.2 to 3 parts by weight of Glycyrrhizae Radix and 1 to 4 parts by weight of Gypsum Fibrosum, more preferably 0.2 to 2 parts by weight of Glycyrrhizae Radix and 1 to 3 parts by weight of Gypsum Fibrosum.

For the medicament of the present invention, while the concentrated extracts or dry extract powders of the mixed crude drugs obtained as described above can be directly used, they can also be used in the form of solid preparations, such as capsules, tablets, granules, fine granules or powders obtained by using a conventional method and adding conventional pharmaceutical additives including excipients and disintegrating agents, for example, lactose, hydroxypropylmethylcellulose, hydroxypropylcellulose, low substituted hydroxypropylcellulose, ethylcellulose, corn starch, crystalline cellulose, carmellose calcium, silicic anhydride, synthetic aluminum silicate and/or magnesium stearate, if necessary.

With respect to the medicament of the present invention, since the crude drugs or extracts thereof have a particular bitter taste, preparations in which such a bitter taste is masked are preferable for oral administration.

As a masking method, a known masking method including the one in which a drug is coated with a coating agent (a film-coating method) or the one in which a drug is dispersed in a base to make a matrix form (a matrix method).

That is, a film coating method can be easily carried out by using coating agents such as gastric or enteric polymers, water-soluble or water-insoluble polymers, to provide a film on the tablets, granules, fine granules or powders obtained as described above.

Specific examples of the above-mentioned coating agents include aminoalkyl methacrylate copolymers, polyvinyl acetyldiethylamino acetate, cellulose acetate phthalate, methacrylic acid copolymers, hydroxypropylcellulose, hydroxylpropylmethylcellulose 2910, methylcellulose and ethylcellulose.

The matrix method can be carried out by kneading crude drugs or extracts thereof with a base comprising a water-insoluble polymer and/or a water-swellable polymer, granulating them to make a matrix form wherein the crude drugs or the extracts thereof are dispersed in the base comprising the above-mentioned polymers, and then preparing them into forms of tablets, granules, fine granules or powders by conventional methods.

Specific examples of the above-mentioned water-insoluble polymers include ethylcellulose and hydroxypropylmethylcellulose phthalate. Further, specific examples of the water-swellable polymers include low substituted hydroxylpropylcellulose, amincalkyl methacrylate copolymers, carmellose calcium, sodium carboxymethyl starch and carboxyvinyl polymers.

Further, pharmaceutical additives including water-soluble polymers such as hydroxypropylcellulose, hydrogenated oils, higher fatty acids such as stearic acid, and/or esters of sucrose fatty acids can be added into the above-mentioned bases, if appropriate.

The medicament of the present invention is used orally in diabetics concomitantly with, or before or after administration of the above-mentioned thiazolidine compound formulated preparation for the purpose of relieving side effects and enhancing hypoglycemic effects of the thiazolidine compound. With regard to the dose in general, the medicament of the present invention at a dose equivalent to 0.5 g to 10 g of an extract powder is orally administered to an adult once a day or divided into twice or three times a day. With regard to thiazolidine compounds, in the case of pioglitazone, 15 to 45 mg is orally administered once a day in general. In the case of rosiglitazone, 4 to 8 mg is orally administered once or divided into twice a day in general.

For the concomitant administration of the medicament of the present invention and a thiazolidine compound, a formulation which contains both a crude drug mixture comprising Ephedrae Herba, Glycyrrhizae Radix and Gypsum Fibrosum each of which is in a form of ground powders, extracts or mixtures of powders and extracts together with a thiazolidine compound can also be prepared and administered.

The present invention will be illustrated in detail in the following Test Examples. As the medicament of the present invention, an extract from a mixture comprising Ephedrae Herba, Glycyrrhizae Radix and Gypsum Fibrosum, and a Bofu-tsusho-san extract which is a crude drug formulation comprising said extract were used. As a thiazolidine compound, pioglitazone hydrochloride was used.

### TEST EXAMPLE 1

### Body Weight Gain Inhibiting Effect

(1) Sample

(a) Control group

(b) A group administered pioglitazone (5 mg/kg/day as pioglitazone)

(c) A group administered the extract powder of Preparation Example 1

(d) A group administered pioglitazone and extract powder of Preparation Example 1

(2) Test Method

(2-1) Method of Administration and Measurement

Divided into groups of 8 mice, 7-week old KKA^{y} mice which were genetically obese diabetic animals were used. Group (a) was fed only on a powder diet (CE-2, produced by Clea Japan, Inc.) for 5 weeks, and Group (b), Group (c) and Group (d) were given pioglitazone, the extract powder of Preparation Example 1, and pioglitazone and the extract powder of Preparation Example 1, respectively, for 5 weeks by preparing a powder diet so that the daily dose of each therein was of the values shown in Table 1. During the period of the test, body weights were measured, and the formulation of the diet were adjusted in accordance with an increase or decrease in body weight to keep the doses per day in Group (b) and Group (d) constant.

TABLE 1

| Group administered | Dose (/day) |
|---|---|
| (a) Control | — |
| (b) Pioglitazone | 5 mg/kg |
| (c) Extract powder of | 1.5 g/kg |
| Preparation Example 1 | |
| (d) Pioglitazone and | 5 mg/kg of pioglitazone and |
| extract powder of Preparation | 1.5 g/kg of extract powder |
| Example 1 | of Preparation Example 1 |

(2-2) Method for Statistical Test

The results were evaluated by comparing the body weight gain observed in each of Groups (a), (b), (c) and (d) (Student's t-test).

(3) Test Results

The results are shown in Table 2.

As clearly shown in Table 2, the body weight gain in the group administered pioglitazone alone (Group (b)) was significantly higher than the control group (Group (a)) both 1 week and 5 weeks after the administration. There was no significant difference in body weight gain between the group administered pioglitazone and the extract powder of Preparation Example 1 in combination (Group (d)) and the control group (Group (a)) at any point of time.

TABLE 2

| | Body weight gain (g) | |
|---|---|---|
| | 1 week after | 5 weeks after |
| Group administered | administration | administration |
| (a) Control | 3.2 ± 0.3 | 9.3 ± 0.4 |
| (b) Pioglitazone | 5.2 ± 0.2** | 11.8 ± 0.6** |
| (c) Extract powder | 2.4 ± 0.2* | 9.0 ± 0.3 NS |
| of Preparation | | |
| Example 1 | | |
| (d) Pioglitazone | 3.9 ± 0.3 NS | 10.6 ± 0.4 NS |
| and extract powder | | |
| of Preparation | | |
| Example 1 | | |

Significant difference from the control group:
*P < 0.05,
**P < 0.01,
NS: No significant difference

### TEST EXAMPLE 2

### Hypoglycemic Effect

(1) Sample

The same samples as Test Example 1 were used.

(2) Test Method

(2-1) Method of Administration and Measurement

Tests were carried out in the same manner as in Test Example 1. The blood was collected 1 week and 5 weeks after commencement of the administration, and the serum was separated to measure the blood glucose levels.

(2-2) Method for Statistical Test

The results were evaluated in the same manner as in Test Example 1, comparing blood glucose levels observed in each group.

(3) Test Results

The results are shown in Table 3.

As clearly shown in Table 3, the blood glucose level of the group administered pioglitazone alone (Group (b)) measured 1 week after the administration was significantly lower than the control group (Group (a)). In the group administered pioglitazone and the extract powder of Preparation Example 1 in combination (Group (d)), a further decrease in blood glucose level was observed when compared with the group administered pioglitazone alone (Group (b)).

With regard to the blood glucose levels 5 weeks after the administration, on the other hand, there was no significant difference between the group administered pioglitazone alone (Group (b)) and the control group (Group (a)) any more. However, a significant decrease in blood glucose level was observed in the group administered pioglitazone and the extract powder of Preparation Example 1 (Group (d)) when compared with the control group (Group (a)).

TABLE 3

| | Blood glucose level (mg/dl) | | |
|---|---|---|---|
| | | 1 week | 5 weeks |
| Group | Before the | after the | after the |
| administered | administration | administration | administration |
| (a) Control | 438 ± 28 | 535 ± 22 | 551 ± 24 |
| (b) | 436 ± 26 | 404 ± 28** | 527 ± 18 NS |
| Pioglitazone | | | |
| (c) Extract | 434 ± 25 | 400 ± 30** | 476 ± 20* |
| powder of | | | |
| Preparation | | | |
| Example 1 | | | |
| (d) | 430 ± 24 | 333 ± 17*** | 416 ± 33** |
| Pioglitazone | | | |
| and extract | | | |
| powder of | | | |
| Preparation | | | |
| Example 1 | | | |

Significant difference from the control group:
*P < 0.05,
**P < 0.01,
***P < 0.001,
NS: No significant difference

### TEST EXAMPLE 3

### Body Weight Gain Inhibiting Effect

(1) Samples

(a) Control group

(b) A group administered pioglitazone (5 mg/kg/day as pioglitazone)

(c) A group administered the extract powder of Preparation Example 2

(d) A group administered pioglitazone and the extract powder of Preparation Example 2

(2) Test Method

(2-1) Method of Administration and Measurement

Divided into groups of 7 mice, 7-week old KKA^{y} mice which were genetically obese diabetic animals were used, and the tests were carried out in the same manner as in Test Example 1, except Group (c) and (d) were given the extract powder of Preparation Example 2, and pioglitazone and the extract powder of Preparation Example 2, respectively, for 4 weeks by preparing the powder diet so that the daily dose each therein was of the values shown in Table 4.

TABLE 4

| Group administered | Dose (/day) |
|---|---|
| (a) Control | — |
| (b) Pioglitazone | 5 mg/kg |
| (c) Extract powder of | 6.4 g/kg |
| Preparation Example 2 | |
| (d) Pioglitazone and | 5 mg/kg of pioglitazone and |
| extract powder of Preparation | 6.4 g/kg of extract powder |
| Example 2 | of Preparation Example 2 |

(2-2) Method for Statistical Test

The results were evaluated in the same manner as in Test Example 1.

(3) Tests Results

The results are shown in Table 5

As clearly shown in Table 5, the body weight gain in the group administered pioglitazone alone (Group (b)) was significantly higher than the control group (Group (a)) both 1 week and 4 weeks after the administration. On the other hand, there was no significant difference in body weight gain between the group administered pioglitazone and the extract powder of Example 2 (Group (d)) and the control group (Group (a)) at any point of time.

TABLE 5

| | Body weight gain (g) | |
|---|---|---|
| | 1 week after | 4 weeks after |
| Group administered | administration | administration |
| (a) Control | 2.3 ± 0.4 | 7.7 ± 0.7 |
| (b) Pioglitazone | 5.3 ± 0.4*** | 10.7 ± 0.5** |
| (c) Extract powder | 1.3 ± 0.4 NS | 6.6 ± 0.3 NS |
| of Preparation | | |
| Example 2 | | |
| (d) Pioglitazone | 1.4 ± 0.5 NS | 7.6 ± 0.6 NS |
| and extract powder | | |
| of Preparation | | |
| Example 2 | | |

Significant difference from the control group:
**P < 0.01,
***P < 0.001,
NS: No significant difference

### TEST EXAMPLE 4

### Hypoglycemic Effect

(1) Samples

The same samples as Test Example 3 were used.

(2) Test Method

(2-1) Method of Administration and Measurement

The test was carried out in the same manner as in Test Example 3. The blood was collected 1 week and 4 weeks after commencement of the administration, and the serum was separated to measure the blood glucose levels.

(2-2) Method for Statistical Test

The results were evaluated in the same manner as in Test Example 2.

(3) Test Results

The results are shown in Table 6.

As clearly shown in Table 6, the blood glucose level of the group administered pioglitazone alone (Group (b)) measured 1 week after the administration was significantly lower than the control group (Group (a)). In the group administered pioglitazone and the extract powder of Preparation Example 2 in combination (Group (d)), a further decrease in blood glucose level) was observed compared with the group administered pioglitazone alone (Group (b)).

With regard to the blood glucose levels 4 weeks after the administration, on the other hand, there was no significant difference between the group administered pioglitazone alone (Group (b)) and the control group (Group (a)) any more. However, a significant decrease in blood glucose level was observed in the group administered pioglitazone and the extract powder of Preparation Example 2 (Group (d)) when compared with the control group (Group (a)).

TABLE 6

| | Blood glucose level (mg/dl) | | |
|---|---|---|---|
| | | 1 week | 4 weeks |
| Group | Before the | after the | after the |
| administered | administration | administration | administration |
| (a) Control | 421 ± 20 | 459 ± 18 | 608 ± 28 |
| (b) | 423 ± 15 | 320 ± 30** | 587 ± 24 NS |
| Pioglitazone | | | |
| (c) Extract | 420 ± 23 | 291 ± 22*** | 457 ± 41* |
| powder of | | | |
| Preparation | | | |
| Example 2 | | | |
| (d) | 422 ± 20 | 221 ± 10*** | 389 ± 18*** |
| Pioglitazone | | | |
| and extract | | | |
| powder of | | | |
| Preparation | | | |
| Example 2 | | | |

Significant difference from the control group:
*P < 0.05,
**P < 0.01,
***P < 0.001,
NS: No significant difference

Hereinafter, the present invention will be illustrated in Preparation Examples and Examples in more detail.

### PREPARATION EXAMPLE 1

To mixed crude drugs comprising 1.0 kg of Ephedrae Herba, 0.5 kg of Glycyrrhizae Radix and 2.5 kg of Gypsum Fibrosum, 40 L of purified water was added and heated at about 100° C. for 1 hour. The decoction was filtered, concentrated under reduced pressure, and then spray dried to obtain the extract powder of Preparation Example 1.

### PREPARATION EXAMPLE 2

Preparation of Extract Powder of Bofu-tsusho-san: To mixed crude drugs comprising 0.24 kg of Angelicae Radix, 0.24 kg of Paeoniae Radix, 0.24 kg of Cnidii Rhizoma, 0.24 kg of Gardeniae Fructus, 0.24 kg of Forsythiae Fructus, 0.24 kg of Menthae Herba, 0.24 kg of Schizonepetae Spica, 0.24 kg of Saposhnikoviae Radix,. 0.24 kg of Ephedrae Herba, 0.4 kg of Atractylodis Rhizoma, 0.4 kg of Platycodi Radix, 0.4 kg of Scutellariae Radix, 0.4 kg of Glycyrrhizae Radix, 0.4 kg of Gypsum Fibrosum, 0.6 kg of Talcum, 0.08 kg of Zingiberis Rhizoma, 0.3 kg of Rhei Rhizoma and 0.15 kg of mirabilite, 52.9 L of purified water was added and heated at about 100° C. for 1 hour. The decoction was filtered, concentrated under reduced pressure, and then spray dried to obtain an extract powder of Bofu-tsusho-san.

### PREPARATION EXAMPLE 3

Preparation of Extract Powder of Goko-to: To mixed crude drugs comprising 0.8 kg of Ephedrae Herba, 0.8 kg of Armeniacae Semen, 0.4 kg of Glycyrrhizae Radix, 2.0 kg of Gypsum Fibrosum and 0.6 kg of Mori Cortex, 46 L of purified water was added and heated at about 100° C. for 1 hour. The decoction was filtered, concentrated under reduced pressure, and then spray dried to obtain an extract powder of Goko-to.

### PREPARATION EXAMPLE 4

Preparation of Extract Powder of Makyo-kanseki-to: To mixed crude drugs comprising 1.2 kg of Ephedrae Herba, 1.2 kg of Armeniacae Semen, 0.6 kg of Glycyrrhizae Radix and 3.0 kg of Gypsum Fibrosum, 60 L of purified water was added and heated at about 100° C. for 1 hour. The decoction was filtered and concentrated under reduced pressure, and then spray dried to obtain an extract powder of Makyo-kanseki-to.

### PREPARATION EXAMPLE 5

Preparation of Extract Powder of Eppi-ka-jutsu-to: To mixed crude drugs comprising 1.2.kg of Ephedrae Herba, 0.4 kg of Glycyrrhizae Radix, 1.6 kg of Gypsum Fibrosum, 0.6 kg of Zizyphi Fructus, 0.8 kg of Atractylodis Rhizoma or Atractylodis Lanceae Rhizoma and 0.2 kg of Zingiberis Rhizoma, 48 L of purified water was added and heated at about 100° C. for 1 hour. The decoction was filtered and concentrated under reduced pressure, and then spray dried to obtain an extract powder of Eppi-ka-jutsu-to.

### EXAMPLE 1

77 parts by weight-of the extract powder of Preparation Example 1, 5 parts by weight of lactose, 14 parts by weight of low substituted hydroxypropylcellulose and 3 parts by weight of hydroxypropylcellulose are sufficiently mixed, and 30 parts by weight of anhydrous ethanol is added, and the mixture is kneaded, granulated by a wet extrusion granulation method, dried and sieved according to grain size to obtain a granulated material. To the thus obtained granulated material, 1 part by weight of magnesium stearate is added and mixed to obtain granule of a side effect-relieving agent and/or a hypoglycemic effect enhancer for thiazolidine compounds of Example 1.

### EXAMPLE 2

77 parts by weight of the extract powder of Bofu-tsusho-san (the extract powder of Preparation Example 2), 5 parts by weight of lactose, 14 parts-by weight of low substituted hydroxypropylcellulose and 3 parts by weight of hydroxypropylcellulose are sufficiently mixed, and 30 parts by weight of anhydrous ethanol is added, and the mixture is kneaded, granulated by a wet extrusion granulation method, dried and sieved according to grain size to obtain a granulated material. To the thus obtained granulated material, 1 part by weight of magnesium stearate is added and mixed to obtain granule of a side effect-relieving agent and/or a hypoglycemic effect enhancer for thiazolidine compounds of Example 2.

### EXAMPLE 3

77 parts by weight of the extract powder of Goko-to (the extract powder of Preparation Example 3), 5 parts by weight of lactose, 14 parts by weight of low substituted hydroxypropylcellulose and 3 parts by weight of hydroxypropylcellulose are sufficiently mixed, and 30 parts by weight of anhydrous ethanol is added, and the mixture is kneaded, granulated by a wet extrusion granulation method, dried and sieved according to grain size to obtain a granulated material. To the thus obtained granulated material, 1 part by weight of magnesium stearate is added and mixed to obtain granule of a side effect-relieving agent and/or a hypoglycemic effect enhancer for thiazolidine compounds of Example 3.

### EXAMPLE 4

77 parts by weight of the extract powder of Makyo-kanseki-to (the extract powder of Preparation Example 4), 5 parts by weight of lactose, 14 parts by weight of low substituted hydroxypropylcellulose and 3 parts by weight of hydroxypropylcellulose are sufficiently mixed, and 30 parts by weight of anhydrous ethanol is added, and the mixture is kneaded, granulated by a wet extrusion granulation method, dried and sieved according grain size to obtain a granulated material. To the thus obtained granulated material, 1 part by weight of magnesium stearate is added and mixed to obtain granule of a side effect-relieving agent and/or a hypoglycemic effect enhancer for thiazolidine compounds of Example 4.

### EXAMPLE 5

77 parts by weight of the extract powder of Eppi-ka-jutsu-to (the extract powder of Preparation Example 5), 5 parts by weight of lactose, 14 parts by weight of low substituted hydroxypropylcellulose and 3 parts by weight of hydroxypropylcellulose are sufficiently mixed, and 30 parts by weight of anhydrous ethanol is added, and the mixture is kneaded, granulated by a wet extrusion granulation method, dried and sieved according to grain size to obtain a granulated material. To the thus obtained granulated material, 1 part by weight of magnesium stearate is added and mixed to obtain granule of a side effect-relieving agent and/or a hypoglycemic effect enhancer for thiazolidine compounds of Example 5.

### INDUSTRIAL APPLICABILITY

The medicament of the present invention, when used with thiazolidine compounds in combination, inhibited body weight gain induced by the thiazolidine compounds (Test Examples 1 and 3) and suppress the reduction of hypoglycemic effect accompanying body weight gain induced by the thiazolidine compounds (Test Examples 2 and 4). The same effects are observed for Goko-to, Makyo-kanseki-to and Eppi-ka-jutsu-to. Accordingly, the medicament of the present invention is useful as a side effect-relieving agent and/or a hypoglycemic effect enhancer for thiazolidine compounds. Further, the use of the medicament of the present invention and the thiazolidine compounds in combination allows the suppression of onset and development of diabetes complications since the blood glucose level is favorably controlled over a long period.

1. A method for relieving a side effect induced by a thiazolidine compound, comprising:
providing a mixture comprising an active ingredient comprising Ephedrae Herba, Glycyrrhizae Radix and Gypsum Fibrosum, each of which is in a form of ground powder, extract or mixture of powder and extract, to a diabetic concomitantly with, or before or after administration of the thiazolidine compound to the diabetic, for relieving the side effect induced by the thiazolidine compound.

2. The method of claim 1, wherein the mixture is selected from Bofu-tsusho-san, Goko-to, Makyo-kanseki-to and Eppika-jutsu-to.

3. The method of claim 1, wherein the mixture is Bofu-tsusho-san.

4. The method of claim 1, wherein the Glycyrrhizae Radix is formulated in an amount of 0.1 to 3 parts by weight and the Gypsum Fibrosum is formulated in an amount of 0.5 to 5 parts by weight on the basis of 1 part by weight of Ephedrae Herba.

5. The method of claim 1, wherein the mixture is coated with a coating agent.

6. The method of claim 1, wherein the mixture is dispersed in a base to make a matrix form.

7. The method of claim 1, wherein the mixture is administered orally.

8. The method of claim 1, wherein the mixture further comprises the thiazolidine compound.

9. The method of claim 1, wherein the thiazolidine compound is selected from pioglitazone, troglitazone, rosiglitazone and pharmaceutically acceptable salts thereof.

10. The method of claim 1, further comprising administering the thiazolidine compound to the diabetic concomitantly with, or before or after the administration of the mixture to the diabetic.

11. A method for enhancing a hypoglycemic effect of a thiazolidine compound, comprising:
providing a mixture comprising an active ingredient comprising Ephedrae Herba, Glycyrrhizae Radix and Gypsum Fibrosum, each of which is in a form of ground powder, extract or mixture of powder and extract, to a diabetic concomitantly with, or before or after administration of the thiazolidine compound to the diabetic, for enhancing the hypoglycemic effect of the thiazolidine compound.

12. The method of claim 11, wherein the mixture is selected from Bofu-tsusho-san, Goko-to, Makyo-kanseki-to and Eppika-jutsu-to.

13. The method of claim 11, wherein the mixture is Bofu-tsusho-san.

14. The method of claim 11, wherein the Glycyrrhizae Radix is formulated in an amount of 0.1 to 3 parts by weight and the Gypsum Fibrosum is formulated in an amount of 0.5.to 5 parts by weight on the basis of 1 part by weight of Ephedrae Herba.

15. The method of claim 11, wherein the mixture is coated with a coating agent or the mixture is dispersed in a base to make a matrix form.

16. The method of claim 11, wherein the mixture is administered orally.

17. The method of claim 11, wherein the mixture further comprises the thiazolidine compound.

18. The method of claim 11, wherein the thiazolidine compound is selected from pioglitazone, troglitazone, rosiglitazone and pharmaceutically acceptable salts thereof.

19. The method of claim 11, further comprising administering the thiazolidine compound to the diabetic concomitantly with, or before or after the administration of the mixture to the diabetic.

20. A method for relieving a side effect induced by a thiazolidine compound and for enhancing a hypoglycemic effect of the thiazolidine compound, comprising:
providing a mixture comprising an active ingredient comprising Ephedrae Herba, Glycyrrhizae Radix and Gypsum Fibrosum, each of which is in a form of ground powder, extract or mixture of powder and extract, to a diabetic concomitantly with, or before or after administration of the thiazolidine compound to the diabetic, for relieving the side effect induced by the thiazolidine compound and for enhancing the hypoglycemic effect of the thiazolidine compound.
